(19) United States
(12) Patent Application Publication
(10) Pub. No.: US 2023/0149899 A1
(43) Pub. Date: May 18, 2023

(54) POROUS SUPERABSORBENT POLYMER MATERIAL FOR MICROALGAE HARVESTING

(71) Applicant: Georgia Tech Research Corporation, Atlanta, GA (US)

(72) Inventors: Xing XIE, Marietta (CN); Wensi Chen, Atlanta (CN)

(21) Appl. No.: 18/054,658

(22) Filed: Nov. 11, 2022

Related U.S. Application Data
(60) Provisional application No. 63/279,223, filed on Nov. 15, 2021.

(51) Int. Cl. B01J 20/28; C12N 1/12; B01J 20/26

(52) U.S. Cl. CPC B01J 20/28021; C12N 1/12; B01J 20/261; B01J 20/28085; B01J 2220/68

(57) ABSTRACT

The present disclosure relates to methods for harvesting microalgae and to porous superabsorbent polymeric materials useful in such processes.

### CROSS REFERENCE TO RELATED APPLICATIONS

This application claims the benefit of U.S. Provisional Application No. 63,279,223, filed Nov. 15, 2021, the entire contents of which is hereby incorporated by reference.

### FIELD OF THE INVENTION

The present invention relates to methods for harvesting microalgae and to porous superabsorbent polymeric materials useful in such processes.

### BACKGROUND OF THE INVENTION

Microalgae are emerging as next-generation sustainable, renewable, and economical sources with extensive application potential to address urgent industrial and agricultural demands. See, e.g., Chisti et al., Biotechnol. Adv., 25(3), 294-306, 2007; Medipally et al., BioMed. Res. Int., 2015, 1, 2015; Khan et al., Microb. Cell Fact., 17(1), 1-21, 2018. Microalgae are rapidly growing organisms with high photosynthetic efficiency, no arable land requirement, and ease of scale-up. See, e.g., Subramanian et al., Biotechnol. Biofuels, 6(1), 1-12, 2013. Attributed to the high contents of lipids and carbohydrates, microalgae as a feedstock are competent and preferable to produce biofuels such as biodiesel and bioethanol. See, e.g., Lam et al., Biotechnol. Adv., 30(3), 673-690, 2012; Kiran et al., Energy Conyers. Manage., 88, 1228-1244, 2014. In addition, microalgae are feasible sources of various high value bioproducts, such as polyunsaturated fatty acids, carotenoids, pigments and antioxidants. See, e.g., Sun et al. Biotechnol. Biofuels, 11(1), 1-23, 2018. To enhance the production of targeted bioproducts, researchers have continually developed and improved microalgae-based technologies, for example, stress manipulation strategies, omics-based technologies, and molecular approaches. See, e.g., Liang et al., Crit. Rev. Food Sci. Nutr., 59(15), 2423-2441, 2019. Furthermore, microalgae-based wastewater treatment that combines wastewater treatment, resource recovery, and biomass production has been proposed and already put into practice, which opens more possibilities for the microalgae industry. See, e.g., Cai et al., Renew. Sust. Energy Rev., 19, 360-369, 2013; Wollmann et al., Eng. Life Sci., 19(12), 860-871, 2019. In recent years, the feasibility of using microalgae in advanced wastewater treatment has been investigated and proven by many studies for its highly efficient removal of nutrients from municipal, agricultural and industrial wastewater and promising production of raw materials for energy and pharmaceutical uses. See, e.g., Christenson et al., Biotech. Adv., 29(6), 686-702, 2011; Wang et al., Bioresource Tech., 222, 485-497, 2016; Li et al., Bioresource Tech., 291, 121934, 2019.

Nevertheless, there are still many obstacles that must be overcome for large-scale industrialization and commercialization of microalgal production and processing. One of the most challenging and crucial issues is microalgae harvesting, which currently causes nearly 20-30% of total biomass production cost and may account for 90% of instrument cost for outdoor microalgae farms. See, e.g., Mathimani et al., Renew. Sust. Energy Rev., 91, 1103-1120, 2018; Amer et al., Bioresource Tech., 102(20), 9350-9359, 2011. The high harvesting cost is because the microalgae usually have small cell size (3-30 μm), dilute cultures (<0.5 g L⁻¹), close-to-water density (1.07-1.14 g cm⁻¹), and electronegative surface charge (−7.5 to −40 mV), which cause difficulties in remove the culture medium and concentrate microalgae. See, e.g., Christenson et al., Biotech. Adv., 29(6), 686-702, 2011; Henderson et al., Water Res., 42 (8-9), 1827-1845, 2008; Ozkan et al., Colloids Surf B, 112, 287-293, 2013.

Conventional and emerging microalgae harvesting methods include physical methods (e.g., centrifugation, membrane filtration, sedimentation, and flotation), chemical methods (e.g., flocculation), electrical methods (e.g., electrophoresis and electrolytic coagulation), and magnetic methods (e.g., Fe₃O₄ based magnetic nanoparticles). See, e.g., Mathimani et al., Renewable Sustainable Energy Rev., 91, 1103-1120, 2018; Wang et al., Algal Res., 9, 178-185, 2015; Singh et al., J. Environ. Manage., 217, 499-508, 2018; Mo et al., Algal Res., 11, 1-12, 2015. Implementation of these methods, however, are hampered by intensive energy consumption, high capital cost, complex operation process, long treatment time, and/or the requirement of chemical addition.

Chlorella vulgaris is one of the most promising microalgae for biofuel production and has been widely studied in recent years. See, e.g., Safi et al. Renewable and Sustainable Energy Reviews, 35, 265-278, 2014. For example, carbohydrate rich Chlorella vulgaris has been used as feedstock for sugar production and bioethanol fermentation, in which nearly 94% glucose yield and up to 88% ethanol conversion efficiency were reported. See, e.g., Ho et al., Bioresour. Technol., 135, 191-198, 2013.

Superabsorbent polymers (SAPs) with a large number of hydrophilic groups are commonly made from synthetic polymers (e.g., sodium polyacrylate and polyacrylamide copolymer) or natural polymers (e.g., cellulose, starch and chitosan). See, e.g., Bao et al., Carbohydr. Polym., 84,76-82, 2011; Guilherme et al., Eur. Polym. 1, 72, 365-385, 2015; Thakur et al., J Cleaner Prod., 198, 143-159, 2018.

SAPs are capable of absorbing and retaining a tremendous amount of water relative to their weight but rejecting large sized components. Thus, a self-driven three-dimensional (3D) filtration process is realized using SAP beads. Unlike normal filtration processes where filtrate passes through either a flat or hollow fiber filter driven by pressure difference, the filtrate spontaneously flows into the SAP beads from all directions. See, e.g., Dou et al., J Mater. Chem. A, 8, 15942-15950, 2020. Due to their excellent water absorption properties, SAP beads have been investigated and applied for purification and concentration of various targets. See, e.g., Iritani et al. Sep. Sci. Technol., 28, 1819-1836, 1993; Wu et al., Sep. Purif. Technol., 239, 116540, 2020; Xie et al., Sci. Rep., 6, 20516, 2016; Yang et al., J Extracell. Vesicles, 10, No. e12074, 2021.

Nevertheless, the swelling of SAP beads can take a long time, especially when the ion concentration of the sample to be treated increases. For example, it took more than 5 hours for commercial polyacrylic SAP beads with a diameter of ˜2 mm to reach a swelling ratio higher than 100 g g⁻¹ in deionized (DI) water and their swelling ratio was only 60 g g⁻¹ even after 12 hours absorption in 1 g L⁻¹ of NaCl solution. See, e.g., del Campo et al., Biotechnol. Bioeng., 110, 3227-3234, 2013; Wei et al., Bioresour. Technol., 249, 713-719, 2018.

Reducing the size of the SAP beads can effectively increase the specific surface area and improve the overall water absorption rate. However, smaller SAP beads are more difficult to separate from the concentrate after use, and their lager specific surface area will decrease the concentration efficiency because of the adsorption of targets on the surface. See, e.g., Xie et al., Sci. Rep., 6, 20516, 2016.

There is therefore a need for efficient cost-effective, and reliable methods for harvesting microalgae for use towards the production of algal extracts.

### SUMMARY OF THE INVENTION

The present invention provides a microalgae harvesting approach using rationally-designed porous superabsorbent polymer (PSAP) beads. The PSAP beads replace regular superabsorbent polymer (SAP) beads. PSAP beads are SAP frameworks distributed with numerous physical pores. See, e.g., FIG. 1. When the PSAP beads are immersed in an aqueous solution, water quickly fills the physical pores and wets the pore surface, which significantly increases the contact area between the polymer network and water molecules and thus improves water absorption rate. The functionality of the PSAP beads for separation is determined by the size of these physical pores, which can be tuned from tens of nanometers to tens of micrometers. Optimal pore size should be small enough to exclude the concentrating targets and at the same time as large as possible for fast water uptake. The pore structure of the beads is therefore optimized to allow fast water absorption but minimize surface attachment of microalgal cells and the consequent biomass loss.

The PSAP beads described herein provide a fast, effective, low-cost, and easily scalable alternative for microalgae harvesting. The microalgae harvesting processes described herein are self-driven microfiltration processes that do not require any complex instruments, such as centrifuges and pumps. Little training is needed for the people who operate the harvesting procedures. In addition, the PSAP beads can be produced in large quantities with low cost.

In a typical harvesting process according to any of the embodiments described herein, the dried PSAP beads are added into the microalgal suspension to be harvested; the beads swell fast to absorb water far beyond their own weight while excluding microalgae outside the beads; thus, microalgae are harvested in the residual concentrated liquid. The optimized pore structure of the PSAP beads allows fast and selective absorption of water, which greatly reduces the processing time without sacrificing harvesting efficiency. In addition, the superior water absorption capacity of the PSAP enables significant concentration degree (e.g., up to ten times more) with only a small amount of the beads. After harvesting, the hydrated beads can be easily separated from the concentrated microalgae and regenerated for multiple reuses.

Accordingly, in one aspect, the present invention relates to a method for harvesting microalgae (e.g., from a medium). In one embodiment, the method comprises
(a) adding a porous superabsorbent polymer bead to the medium;
(b) allowing water in the medium to absorb into pores in the porous superabsorbent polymer beads to form hydrated beads; and
(c) separating the microalgae from the hydrated beads, thereby harvesting the microalgae.

In another aspect, the present invention relates to a method for increasing the concentration of microalgae (e.g., in a medium). In one embodiment, the methods comprises
(a) adding a porous superabsorbent polymer bead to the medium;
(b) allowing water in the medium to absorb into pores in the porous superabsorbent polymer beads to form hydrated beads, thereby increasing the concentration of microalgae in the medium; and
(c) optionally, separating the microalgae from the hydrated beads.

In certain embodiments of any of the methods described herein, the microalgae is a marine microalgae.

In one embodiment of any of the methods described herein, the microalgae can grow in seawater provided directly from the ocean.

In other embodiments of any of the methods described herein, the microalgae is a freshwater or brackish water microalgae. In other embodiments, the microalgae exist as single cells, as groups, or in chains.

In other embodiments of any of the methods described herein, the microalgae is Chlorella vulgaris, Isochrysis sp Pseudoisochrysis sp., Dicrateria sp., Monochrysis sp., Tetraselmis sp., Pyramimonas sp., Micromonas sp., Chroomonas sp., Cryptomonas sp., Rhodomonas sp., Chlamydomonas sp., Chlorococcum sp., Olisthodiscus sp., Carteria sp., Dunaliella sp., Spirulina sp., Haematococcus sp., Rhodella sp., Arthrospira maxima, or Nannochloropsis sp, or any combination thereof.

In one embodiment of any of the methods described herein, the microalgae is Chlorella vulgaris.

In some embodiments of any of the methods described herein, the microalgae (such as Chlorella vulgaris) may comprise genetic differences from wild-type microalgae or microalgae found in nature.

In another embodiment of any of the methods described herein, the process further comprises a step of drying the microalgae after separation from the hydrated beads.

In another embodiment of any of the methods described herein, in step (b) microalgae are excluded from the pores of the bead.

In another embodiment of any of the methods described herein, in step (b) microalgae are concentrated in the medium.

In another embodiment of any of the methods described herein, the medium is a microalgal suspension. In another embodiment of any of the methods described herein, the medium is a liquid comprising non-concentrated microalgae. In another embodiment of any of the methods described herein, the medium is a liquid, e.g., water, comprising microalgae (e.g., non-concentrated microalgae).

In certain embodiments of any of the methods described herein, the water is saltwater, freshwater, or brackish water. In other embodiments of any of the methods described herein, the water is seawater. In one embodiment of any of the methods described herein, the water is pumped from the ocean.

In certain embodiments of any of the methods described herein, the water has a salinity between about 0.01% and about 5%. In other embodiments, the water has a salinity between about 0.01% and about 0.5%, such as between about 0.5% and about 1.0%, between about 1.0% and about 1.5%, between about 1.5% and about 2.0%, between about 2.0% and about 2.5%, between about 2.5% and about 3.0%, between about 3.0% and about 3.5%, between about 3.5% and about 4.0%, between about 4.0% and about 4.5%, or between about 4.5% and about 5.0%. In one embodiment, the water has a salinity of about 0.1%. In one embodiment, the water has a salinity of about 2.2%. In one embodiment, the water has a salinity of about 3.5%.

In certain embodiment of any of the methods described herein, the porous superabsorbent polymer comprises a copolymer.

In one embodiment of any of the methods described herein, the copolymer comprises one or more monomers selected from ionic monomers, non-ionic monomers, and any combination thereof.

In one embodiment of any of the methods described herein, the ionic monomer is selected from sodium acrylate, acrylic acid, potassium acrylate, itaconic acid, sodium itaconate, potassium itaconate, and any combination thereof.

In one embodiment of any of the methods described herein, the non-ionic monomer is acrylamide.

In certain embodiments, the pores in the superabsorbent polymer beads are between about 0.001 and about 10 micrometers, such as between about 0.001 and about 0.01 micrometers, between about 0.01 and about 0.05 micrometers, between about 0.05 and about 1 micrometers, or between about 1 and about 10 micrometers. For example, in one embodiment, the pores in the superabsorbent polymer beads are less than about 1 micrometer.

In one embodiment, the water absorbency of the superabsorbent polymer beads is between about 10 and about 150 g g⁻¹, such as between about 50 and about 100 g g⁻¹. In one embodiment, the water absorbency of the superabsorbent polymer beads is greater than about 50 g g⁻¹, such as greater than about 60, g g⁻¹, greater than about 65 g g⁻¹ or greater than about 70 g g⁻¹.

Any of the porous superabsorbent polymer beads described herein may be prepared by a process that comprises reacting one or more ionic monomers, and/or one or more non-ionic monomers, or any combination thereof, with a porogen, optionally in the presence of a cross-linker.

Suitable porogens for use in preparing the porous superabsorbent polymer beads according to any embodiment described herein include, but are not limited to, polyethylene glycol (PEG), and polyvinyl alcohol. In one embodiment, the PEG has a molecular weight of between about 200 and about 20,000 (e.g., PEG 6000).

In one embodiment, the amount of porogen present in the monomer solution during preparation of the porous superabsorbent polymer beads is an amount between about 1 wt. % and about 10 wt. %, such as between about 2.5 wt. % and about 7.5 wt. %, relative. In certain embodiments, the porogen is present in an amount of about 2.5 wt. %, about 5 wt. % or about 7.5 wt. %. %. In one embodiment, the porogen is present in an amount of about 5 wt. %.

In one embodiment, the porous superabsorbent polymer beads are washed (e.g., with 95% ethanol) to remove excess porogen.

In one embodiment of any of the methods described herein, the swelling ratio of the porous superabsorbent polymer beads is between about 1 g g⁻¹ and about 500 g g⁻¹, such as between about 1 g g⁻¹ and about 100 g g⁻¹, between about 100 g g⁻¹ and about 200 g g⁻¹ between about 200 g g⁻¹ and about 300 g g⁻¹, between about 300 g g⁻¹ and about 400 g g⁻¹, or between about 400 g g⁻¹ and about 500 g g⁻¹. In certain embodiments, the swelling ratio of the superabsorbent polymer beads is about 56, about 70, or about 120 g g⁻¹.

In one embodiment of any of the methods described herein, the initial biomass density of the medium is between about 0.2 g L⁻¹ and about 70 g L⁻¹, such as between about 0.2 and about 0.6 g L⁻¹.

In one embodiment of any of the methods described herein, the final biomass density of the medium is between about 0.4 g L⁻¹ and about 150 g L⁻¹.

In one embodiment of any of the methods described herein, the efficiency of the process (harvesting efficiency) is greater than about 70%, such as greater than about 75%, greater than about 80%, greater than about 85%, greater than about 90%, greater than about 95%, greater than about 98%, or greater than about 99%.

In one embodiment of any of the methods described herein, the loss of biomass during the process is less than about 30%, such as greater less about 25%, less than about 20%, less than about 15%, less than about 10%, less than about 5%, less than about 2%, or less than about 1%.

In one embodiment of any of the methods described herein, the method further comprises (d) dewatering the hydrated beads (e.g., by drying at, for example 60° C.), then repeating steps (a), (b) and (c) one or more times (e.g., the beads are dried before each water absorption cycle),

In one embodiment of any of the methods described herein, steps (a), (b) and (c) are repeated, one, two, three, four or five times.

In one embodiment of any of the methods described herein, the pH of the medium is between about 6 and about 10, such as between about 6 and about 7, between about 7 and about 8, between about 8 and about 9, or between about 9 and about 10.

In one embodiment of any of the methods described herein, the method is conducted at a temperature raging between about 10° C. and about 30° C., such as between about 10° C. and about 15° C., between about 15° C. and about 20° C., between about 20° C. and about 25° C., or between about 25° C. and about 30° C.

In another aspect, the present invention relates to a porous superabsorbent polymer bead (e.g., for use in microalgae harvesting), wherein the bead exhibits one or more (such as one, two, three or four) of the following properties:
(i) a pore size between about 0.001 and about 10 micrometers,
(ii) a water absorbency between about 10 and about 150 g
(iii) a swelling ratio between about 1 g g⁻¹ and about 500 g g⁻¹,
(iv) a microalgae harvesting efficiency of greater than about 70%.

### BRIEF DESCRIPTION OF THE DRAWINGS

FIG. 1 is a schematic of the PSAPs described herein for microalgae harvesting. The PSAPs have micrometer-sized water channels allowing absorption of water but excluding microalgal cells.

FIG. 2A is a schematic of the PSAP fabrication and pore generation process.

FIG. 2B shows scanning electron microscopy (SEM) images of PSAP beads prepared with different amounts of porogen (2.5, 5.0, or 7.5 wt. % polyethylene glycol (PEG)).

FIG. 3A shows the swelling ratio of SAP and PSAP beads prepared with different amounts of porogen in BG-11 medium (no PEG for SAP beads and 2.5-7.5 wt. % of PEG for PSAP beads).

FIG. 3B shows optical images of the SAP and PSAP beads during the swelling process under digital microscopy.

FIG. 4A shows the swelling ratio of the beads prepared with different amounts of porogen (2.5-7.5 wt. % PEG) in microalgal suspension.

FIG. 4B shows the microalgae harvesting efficiency of the PSAP beads. The concentration factor (a comparison of the volume of the initial microalgal suspension and the final concentrated microalgal suspension) was set at 2 for all harvesting experiments.

FIG. 4C shows digital, differential interference contrast (DIC), and fluorescence (FL) images of the PSAP beads after microalgae harvesting. The initial concentration of the microalgal suspension used was 0.2 g L⁻¹.

FIG. 5A shows microalgal harvesting efficiency and concertation factor at different PSAP dosages.

FIG. 5B shows the swelling ratio of the hydrated beads and microalgae biomass density after treatment at different PSAP dosages.

FIG. 5C shows flow cytometry plots of stained microalgal samples before and after PSAP treatment. (CF=concentration factor). The PSAP beads used were all prepared using 5.0 wt. % PEG as the porogen. The initial microalgal concentration was 0.2 g L⁻¹ for the harvesting experiments.

FIG. 6A shows the swelling ratio of PSAP beads and concentration factors of microalgal suspension.

FIG. 6B shows the microalgae harvesting efficiency of the PSAP beads. The inset image in FIG. 6B shows the PSAP beads in microalgal suspension with different initial concentrations (1-70 g L⁻¹). The PSAP beads used were all prepared using 5.0 wt. % PEG as porogen. The PSAP dosage was around 8 g L⁻¹ for the harvesting experiments.

FIG. 7A shows absorption/desorption cycles of the PSAP beads in microalgal suspension or under heating dewatering.

FIG. 7B shows the microalgae harvesting performance of the reclaimed PSAP beads. The PSAP beads used were all prepared with 5.0 wt. % PEG as porogen. The initial microalgal concentration was 0.2 g L⁻¹ for the harvesting experiments. The PSAP beads were washed with DI water after five cycles of reuse.

FIG. 8 shows the correlation between optical density (OD) at 685 nm (OD₆₈₅) and microalgal biomass density (dry cell weight). The biomass density was measured by filtering 25 mL of microalgal suspension through a microfiltration membrane filter (0.2 μm pore size). The loaded filter was dried at 60° C. until the weight is invariant. The weight of the blank filter was subtracted from that of the loaded filter to calculate the dry cell weight of microalgae.

FIG. 9 shows optical images of the PSAP beads. The dried PSAP products have an average diameter of ˜1.5 mm.

FIG. 10 shows SEM images of SAP beads. The SAP beads were prepared by the precursor without the addition of a porogen.

FIG. 11 shows microalgae harvesting performance of the SAP beads. The SAP dosage was around 8 g L⁻¹ and the treatment time was set at 30 minutes. The final concentration factor was around 1.3 when the initial microalgal concentration was in range of 0.2-0.6 g L⁻¹.

### DETAILED DESCRIPTION OF THE INVENTION

As used herein the following definitions shall apply unless otherwise indicated.

As used herein, the term “microalgae” refers to microscopic, unicellular algae that can exist individually or in groups.

As used herein, the term “porous superabsorbent polymer” refers to a polymer which has tunable pore structure and water-absorbing ability.

As used herein, the terms “water absorbency” and “swelling ratio” refer to the mass of the water a polymer absorbed relative to its own mass.

As used herein, the term “harvesting efficiency” refers to the efficiency of the mass of microalgae collected after harvesting process compared with its original amount.

### Preparation and Characterization of PSAP Beads

Porous polymers may be prepared using conventional methods (e.g., suspension, dispersion, precipitation, and multistage polymerization) and emerging methods (e.g., membrane emulsification and microfluidic technique). See, e.g., Gokmen et al., Prog. Polymer Sci., 37, 365-405, 2012; Wu et al., Chem. Rev., 112, 3659-4015, 2012.

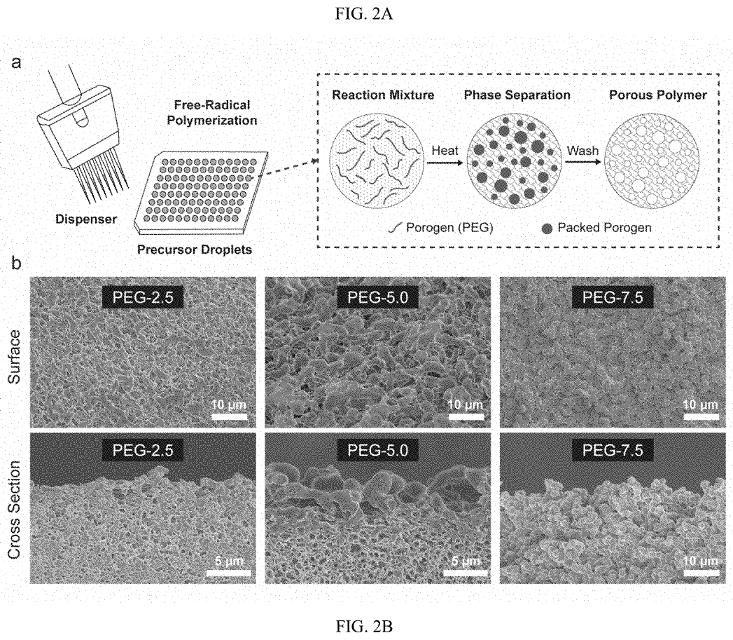

In the present invention, PSAP beads were fabricated with desired micrometer-sized pores via polymerization-induced phase separation. See, e.g., Chen et al., ACS Mat. Lett., 2, 1545-1554, 2020. As shown in FIG. 2A, the linear polymeric porogen, PEG, is uniformly dispersed in the precursor (i.e., the solution containing all monomers and porogen) droplet but not miscible with monomers. The thermal dissociation of the initiator (such as, e.g., ammonium persulfate) generates free radicals and initiates a series of polymerization reactions. Next, phase separation between the packed porogen and crosslinked polymer network occurs, which subsequently yield the porous structure. See, e.g., Guo et al., Angew. Chem. Int. Ed., 53, 7504-7509, 2014; Guo et al., React. Funct. Polym., 91-92, 77-84, 2015. Thus, the pore distribution in the PSAP beads mainly depends on the phase separation process and can be tuned by adjusting polymerization conditions and porogen amount.

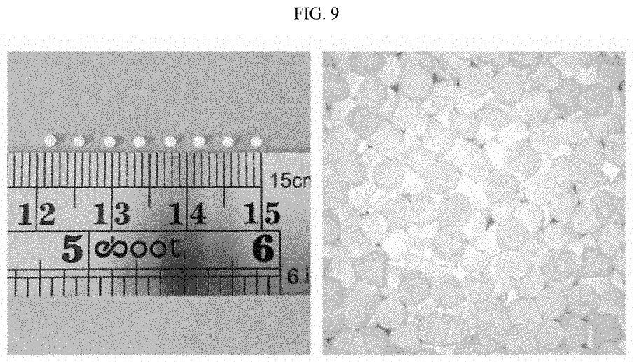

To obtain PSAP beads with optimal pore structure and swelling properties, precursors with different amounts of porogen (e.g., PEG) were used, including, e.g., 2.5, 5.0, and 7.5 wt. % PEG, and the PSAP beads obtained are labeled as PEG-2.5, PEG-5.0, and PEG-7.5, respectively. The dried PSAP products are white-colored beads with an average diameter of around 1.5 mm (See FIG. 9). The surface and cross section morphology of the as-synthesized beads is shown in FIG. 2B. The pore structure of the PSAP beads is highly affected by the porogen concentration. When the PEG amount is 2.5 wt. %, the surface of the beads is rough and irregular with numerous visible small pores. Meanwhile, a uniform and highly porous sponge-like structure is observed for the cross section of the beads. Most of the pores in the beads are around 0.1-0.5 μm in diameter. As the amount of porogen (e.g., PEG) increases to 5.0 wt. %, both the porosity and pore size of the beads increase. The beads covered with an uneven skin layer (less than 5 μm) have continuous porous structure inside. The largest pores of such beads can have a diameter of around 1 μm. When the porogen (e.g., PEG) amount further increases to 7.5 wt. %, the bead morphology changes significantly. The resulting PSAP beads have a relatively sparse dendritic structure, in which the gaps regarded as amorphous pores form interconnected channels. This morphology transformation is because the PEG amount affects porogen aggregation and interaction between the gradually aggregated porogen and growing polymer chain, which in turn affects the phase separation and pore generation. At a lower PEG amount, the polymerization induced phase separation occurs after the gel point, which results in a continuous polymer network with small pores. When the PEG amount reaches a higher level, the increased repulsion between the polymer segments and aqueous PEG solution triggers the phase separation prior to the gel point. Therefore, the porogenic nuclei continue to aggregate and grow, eventually resulting in a discontinuous polymer network with a higher porosity and larger pores. As for PEG-5.0 beads, the porogen amount (5 wt. %) is at the transitional stage between the two phase separation mechanisms, which may cause the asymmetrical structure and a skin layer with relatively larger pores on the bead surface.

### Swelling Behavior of PSAP Beads

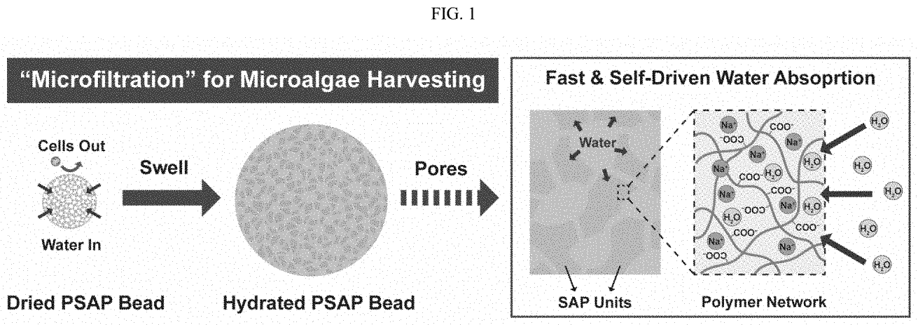

The swelling behavior of the PSAP beads was studied using microalgae cultivation medium, BG-11 medium. See, e.g., Liu et al., Environ. Sci.: Nano, 7, 2021-2031, 2020. Since the precursor contains a large amount of ionic monomer, lots of ionic side groups are bonded to the polymer backbone after polymerization. The resulting PSAP beads have high water affinity and high permeability. When the dried beads are in contact with an aqueous solution, the water molecules attack the bead surface and quickly penetrate into the inner pores due to the capillary effect. As the polymer network swells, the pores inside the beads are enlarged and gradually filled with water. Thus, the swelling process of the beads is spontaneous and does not need additional driving forces. The highly porous structure of the PSAP beads greatly increases the specific surface area of the polymer, enhances its contact with water molecules, and also provides extra volume for water retention (See FIG. 1). Therefore, both the water absorption rate and capacity of the PSAP beads are significantly improved compared with non-porous polymer material.

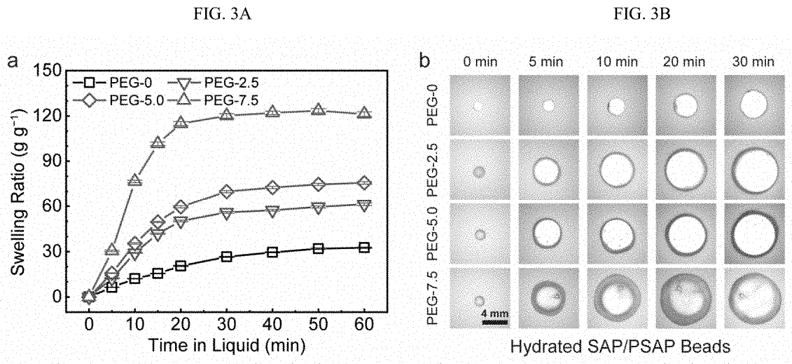

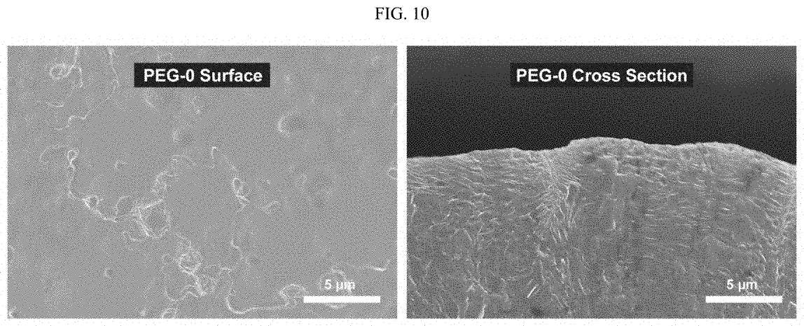

To investigate the effects of pore structure on bead swelling, the swelling kinetics of the SAP beads and PSAP beads in BG-11 medium was monitored. The SAP and PSAP beads have the same chemical composition for the polymer network, except for the difference in the pore structures (See FIG. 2B and FIG. 10). As shown in FIG. 3A, the non-porous SAP beads exhibit a slow expansion in the testing medium due to their dense polymer structure, which have a swelling ratio of only 27 g g⁻¹ after 30 minutes and 33 g g⁻¹ even after 60 minutes absorption. In contrast, the water absorption rate of the PSAP beads significantly increases compared with the SAP beads. These results illustrate that there are three stages in the PSAP swelling process: initial rapid swelling (pore expansion), then slow swelling (polymer chain relaxation), and final saturation state (osmotic pressure equilibrium). The time required for each swelling phase is determined by the pore structure of the PSAP beads (See FIG. 2B). The beads with a higher PEG amount have a faster swelling rate and can achieve a higher swelling ratio at the same time due to their higher porosity and larger pore size. The swelling ratios of the beads with 2.5, 5.0, and 7.5 wt. % PEG after 30 min absorption in BG-11 medium are 56, 70, and 120 g g−1, respectively (See FIG. 3A). The optical images of the beads in FIG. 3B indicate the uniform expansion of the PSAP during the swelling process. The pore structure difference also affects the optical and mechanical properties of the beads after swelling. Similar to the regular SAP beads, the swollen PSAP beads with 2.5 and 5.0 wt. % PEG are transparent and elastic. In contrast, the beads with 7.5 wt. % PEG are translucent and ductile after swelling, which may be relatively fragile and more difficult to separate from the treated solution.

### Microalgae Harvesting Performance

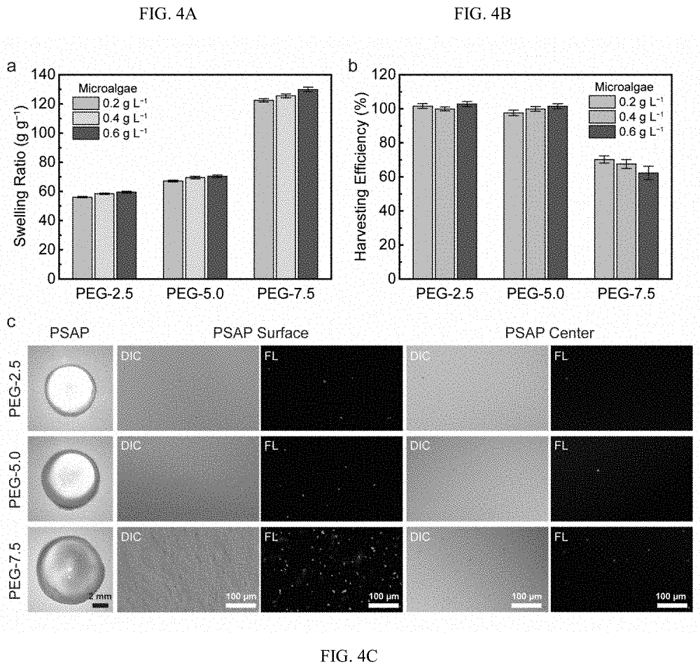

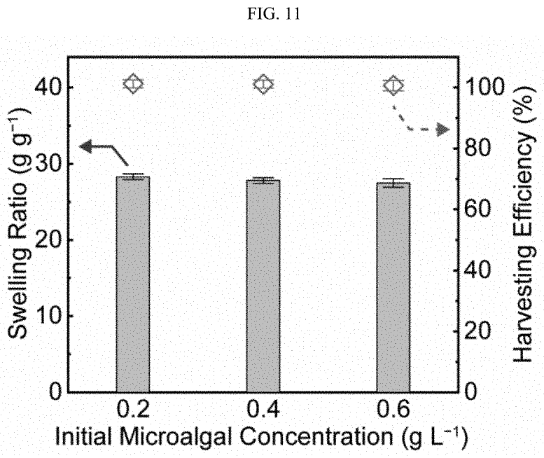

According to the rational design, the optimal PSAP beads should achieve both a high swelling capacity, such that only a small number of beads is required for high capacity concentration and a high harvesting efficiency that the loss of microalgae is minimized during the concentration process. To evaluate the microalgae harvesting performance, the PSAP beads were used to concentrate the model microalga, Chlorella vulgaris, which is a unicellular and spherically shaped green alga with a size of 3-5 μm. FIG. 4A shows the swelling ratio of the PSAP beads in microalgal suspensions with different initial biomass densities (0.2-0.6 g L⁻¹ in dry cell weight). The results show that the swelling properties of the PSAP beads in microalgal suspension are similar to that in BG-11 medium (See FIG. 2A). The swelling capacity of the beads is mainly determined by the pore structure, which increases as the PEG amount increases. For example, when the microalgal concentration is 0.2 g L⁻¹, the swelling ratios of the beads with 2.5, 5.0, and 7.5 wt. % PEG are 56, 67, and 123 g g⁻¹, respectively. Meanwhile, the performance of the SAP beads for microalgae harvesting was also tested as a control group (See FIG. 11). The swelling ratio of the SAP beads is relatively low in the microalgal suspension due to the slow liquid diffusion in the dense and non-porous polymer network, which is only 27 g g⁻¹ after the same treatment time at a microalgal concentration of 0.2 g L⁻¹ (See FIG. 11).

Based on the swelling properties results, the PSAP beads were applied to harvest microalgae, and spectrophotometry was used to measure microalgae concertation, thereby analyzing harvesting efficiency. The treatment time was 30 minutes for all harvesting experiments. The concentration factor was set at around 2, which means the liquid volume should be reduced to half after treatment and the final microalgae concentration would be doubled. The PSAP beads are negatively charged because of the carboxylate groups introduced by co-polymerization of the ionic monomer. The negative surface charge of the beads results in an electrostatic repulsion toward negatively charged microalgal cells during the harvesting at circum-neutral pH conditions. The results shown in FIG. 4B illustrate that the PSAP beads with a lower PEG amount (2.5-5.0 wt. %) can achieve nearly complete recovery of microalgae as the non-porous SAP beads (See FIG. 11), which indicates almost no microalgal cells are lost during the harvesting. Meanwhile, the initial microalgae concentration has negligible effects on the harvesting efficiency. However, when the PEG amount is 7.5 wt. %, the harvesting efficiency significantly decreases, which is only 70% at an initial microalgae concentration of 0.2 g L⁻¹. As the microalgae concentration increases to 0.6 g L⁻¹, the harvesting efficiency of PSAP beads with 7.5 wt. % PEG further decreases to 62%.

Without wishing to be bound by theory, the decrease in harvesting efficiency may be caused by more microalgal cells attaching to the PSAP beads with higher porosity and left on the beads after treatment. Both differential interference contrast (DIC) and fluorescence microscopy were used to observe and visualize the distribution of microalgal cells on bead surface or inside beads after treatment. As shown in FIG. 4C, the PSAP beads with 2.5 or 5.0 wt. % PEG are clean and transparent after harvesting microalgae, while the beads with 7.5 wt. % PEG show slight green due to microalgae remaining on the bead surface. From the DIC images, the surface of the beads with a lower PEG amount (2.5-5.0 wt. %) is smooth and even. Meanwhile, the beads with a higher PEG amount (7.5 wt. %) have a relatively rough and uneven surface, which is more likely to trap particles. The endogenous fluorescence from the microalgal cells is detected by fluorescence microscopy to distinguish cells from the background clearly. The results shown in FIG. 4C show that almost no microalgal cells attach or enter the beads with 2.5 or 5.0 wt. % PEG. On the other hand, for the beads with 7.5 wt. % PEG, although the microalgal cells cannot enter the beads due to the pore size limitation, the surface attachment significantly increases, which results in biomass loss during separation and thus a lower harvesting efficiency. This phenomenon is consistent with the previous results of microalgae harvesting performance testing (See FIG. 4B).

In order to achieve effective and efficient microalgae harvesting at the lowest possible cost, PSAP beads were prepared with 5.0 wt. % PEG and selected due to their excellent swelling properties and optimal pore structure. The selected PSAP beads have a highly porous structure and thus large specific surface area, resulting in a much higher rate of water absorption than non-porous polymer. It takes only 30 minutes for such beads to reach their water absorption capacity, while it may require several hours for normal SAPs to achieve a similar swelling level. In addition, both the porosity and pore size of the PSAP beads with 5.0 wt. % are moderate so that the water absorption capacity is sufficient while the adsorption of microalgal cells onto the beads is minimized to avoid possible biomass loss after harvesting.

### Process Optimization of Microalgae Harvesting

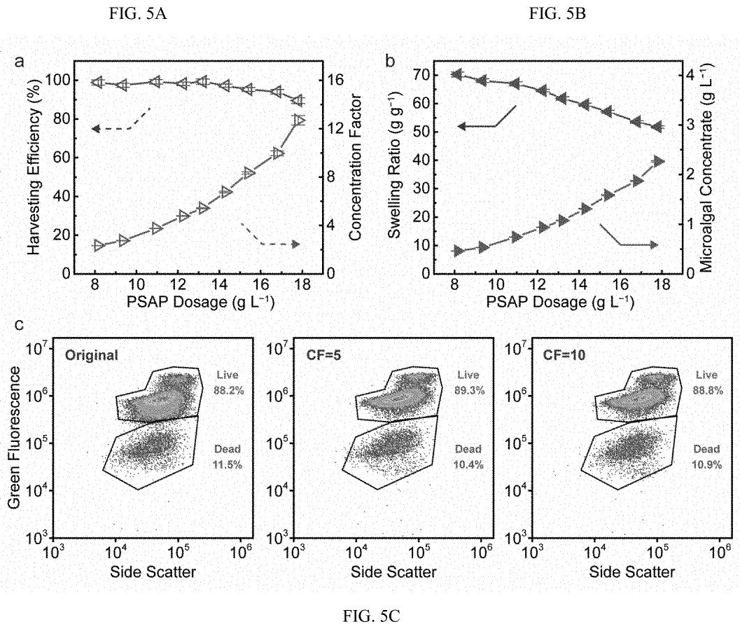

The harvesting process using PSAP beads with 5.0 wt. % PEG was further studied and optimized. The concentration factor, namely the ratio of the sample volume before and after treatment, is an important parameter in the microalgae harvesting process. For a certain amount of microalgal suspension, an increased PSAP dosage should result in a higher concentration degree due to the continuous decrease in residual liquid volume. As shown in FIG. 5A, for 1 L of microalgal suspension, only 8 g of beads leads to a two times concentration (final volume of 0.5 L), 12 g of beads results in a five times concentration (final volume of 0.2 L), and 18 g of beads achieves a concentration factor up to 13 (final volume of ˜0.08 L). The results indicate that the microalgae harvesting efficiency is almost 100% when the concentration factor is not higher than 6 (See FIG. 5A). As the concentration factor increases, the harvesting efficiency slightly decreases, (95% at a concentration factor of 8 and 89% at a concentration factor of 13). FIG. 5B shows the swelling ratio of the PSAP beads and biomass density of the concentrated microalgae. The swelling ratio after the same treatment time slightly decreases as the PSAP dosage increases. This may be because at a higher PSAP dosage, the more concentrated microalgal cells may more easily block the pore channels on the PSAP surface. Then both the water diffusion inside the pore structures and polymer chain relaxation are slowed down, resulting in an overall lower swelling rate. Despite this, the swelling ratio of the beads after treatment is still higher than 50 g g⁻¹ even when the biomass density of the concentrated microalgae is over 2 g L⁻¹.

The decline of recovery efficiency at higher concentration factors is mainly caused by losses of liquid during the separation of beads from the concentrated microalgae. For example, when the PSAP dosage increases, the entrainment of liquid between the hydrated beads increases, which is difficult to separate or collect and results in loss of microalgae. At a higher concentration factor, the higher viscosity of the concentrated microalgal suspension may increase the surface attachment of microalgal cells to the beads and result in lower harvesting efficiency. Considering all these effects, when the initial microalgal concentration is 0.2 g L⁻¹, an optimized concentration factor for microalgae harvesting should be about 4-6 to avoid disadvantageous loss of biomass for a single-stage operation. Under such conditions, the PSAP beads can achieve an effective and low-cost concentration with the harvesting efficiency higher than 95%, which is comparable to or even better than the performance of energy-intensive harvesting methods such as centrifugation. See, e.g., Singh et al., J. Environ. Manage., 217, 499-508. The optimal concentration factor may vary depending on the initial microalgal concentration. For example, a relatively lower concentration factor may be preferred to maintain a high harvesting efficiency at higher initial microalgal concentrations due to the increased cell attachment.

The fast and efficient microalgae harvesting methods using PSAP beads, as described herein, also have several additional advantages, such as simple operation, mild conditions, nontoxicity, and free of residues. Thus, the viability of the microalgal cultures should not be affected after treatment. To demonstrate the viability, flow cytometry combined with cell staining was used to detect and identify live and dead cells in microalgal samples. Because of the difference in permeability, live microalgae with intact cell membranes exhibited significantly enhanced green fluorescence, while dead microalgae with damaged cell membranes exhibited weak green fluorescence under the same excitation conditions. FIG. 5C shows the cytograms of green fluorescence versus side scatter for microalgal cells before and after harvesting by PSAP beads, in which live and dead cells are differentiated by fluorescence signal intensity. As can be seen, there is no significant difference between the microalgal cells before and after PSAP treatment. These results suggest that the PSAP harvesting will not damage the cell membrane integrity and hence preserve the viability of microalgae.

In some cases, the microalgal concentration may still not be high enough after a single concentration treatment. For example, it requires an extremely high biomass density for effective processing in direct oil extraction and biogas fermentation (30-100 g L⁻¹ in dry weight). See, e.g., del Campo et al., Biotechnol. Bioeng., 110, 3227-3234, 2013; Wei et al., Bioresour. Technol., 249, 713-719, 2018. Under this circumstance, multistage harvesting may be required in order to obtain desired concentrated products.

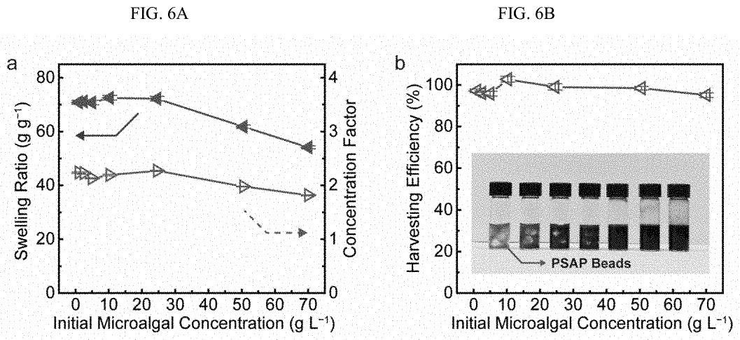

Accordingly, the harvesting performance of the PSAP beads in microalgal suspensions with a relatively high initial concentration was investigated. As shown in FIG. 6A, within a certain range (e.g., when the initial microalgal concentration is below 30 g/L), the water absorption rate of the PSAP beads and the associated concentration factor are not affected by the initial microalgal concentration. At the same PSAP dosage (˜8 g L⁻¹), the swelling ratio maintains at a high level (˜70 g g⁻¹) within the short treatment time (30 minutes) when the initial microalgal concentration increases from 1 to 25 g L⁻¹. As the initial microalgal concentration further increases, the swelling ratio slightly decreases due to the higher viscosity of the microalgal suspension and slower water absorption rate of the beads. Despite this, the PSAP swelling ratio is still higher than 50 g g⁻¹ even when the initial microalgal concentration is as high as 70 g L⁻¹. Meanwhile, the harvesting efficiency is above 95% regardless of the biomass density, which indicates the cell attachment is still negligible at such concentration degrees although the final biomass density reaches higher than 120 g L⁻¹ (See FIG. 6B). These results suggest that microalgal culture could be concentrated for more than 600 times from an initial concentration of 0.2 g L⁻¹ to a final concentration higher than 120 g L⁻¹ with minimal biomass loss through multistage PSAP treatments. The resulting high-concentration microalgae culture can be used directly for the extraction of biofuels and other valuable bioproducts.

### Regeneration and Reuse of PSAP Beads

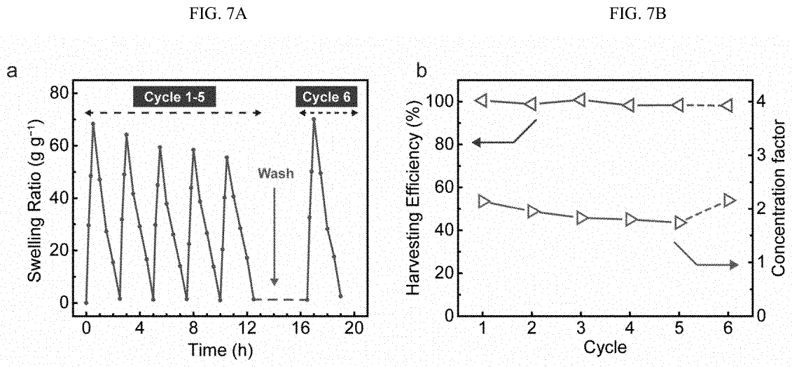

To achieve sustainable microalgae harvesting processes, the possibility of reusing the PSAP beads was investigated. To regenerate the PSAP beads, the hydrated beads after microalgae harvesting were heated in an oven to evaporate absorbed water. The reclaimed beads were then reused to concentrate and harvest microalgae. The absorption-desorption process of the PSAP beads was controlled under the same conditions, in which the absorption time is 30 minutes at 22° C. and the desorption time was 2 hours at 60° C. for each cycle. FIG. 7A shows the changes in the swelling ratio of the PSAP beads during the absorption-desorption cycles. The results show a reduction in the maximal absorbency of the beads, which decreases from 68 to 55 g g⁻¹ after five cycles. This may be because during the microalgae harvesting, ions in the medium enter the beads along with water molecules and remain in the beads after dewatering, which may affect the relaxation of polymer chains and diminish their further absorption capacity. For microalgae harvesting performance, as shown in FIG. 7B, the concentration degree decreases slightly during the reuse of the beads due to their decreased swelling capacity. However, the harvesting efficiency remains higher than 95% even after five cycles, which illustrates the reclaimed PSAP beads still maintain intact structure to effectively absorb water but exclude microalgal cells. To recover the water absorption capacity, the PSAP beads were soaked and washed with DI water to remove absorbed salts on the polymer surface or inside the polymer chains after five cycles of reuse. As shown in FIG. 7A, washing with DI water yields excellent results and brings the maximal swelling ratio back to 70 g Meanwhile, the concentration factor also returns to the level using the original PSAP beads while the harvesting efficiency is still close to 100% (See FIG. 7B).

### EXAMPLES

### Materials and Methods

### Microalgae Cultivation and Biomass Measurement

The microalgae used in this work, Chlorella vulgaris, was obtained from Carolina Biological Supply Company (Burlington, N.C.). The microalgae were cultivated using BG-11 culture medium. The composition of BG-11 medium for microalgae cultivation is shown in the table below.

| | Concentration | | Concentration |
|---|---|---|---|
| Compound | (mM) | Compound | (mM) |
| H₃BO₃ | 46 | NaNO₃ | 0.23 |
| MnCl₂ 4H₂O | 9 | K₂HPO₄ | 0.3 |
| ZnSO₄ 7H₂O | 0.77 | MgSO₄ 7H₂O | 0.24 |
| Na₂MoO₄ 2H₂O | 1.6 | CaCl₂ 2H₂O | 0.031 |
| CuSO₄ 5H₂O | 0.3 | Citric Acid H₂O | 0.021 |
| Co(NO₃)₂ 6H₂O | 0.17 | Ferric Ammonium | 0.0027 |
| | | Citrate | |
| NaNO₃ | 17.6 | Na₂EDTA 2H₂O | 0.19 |

The as-prepared BG-11 medium had a pH of 7.76 and conductivity of 2360 μS cm⁻¹.

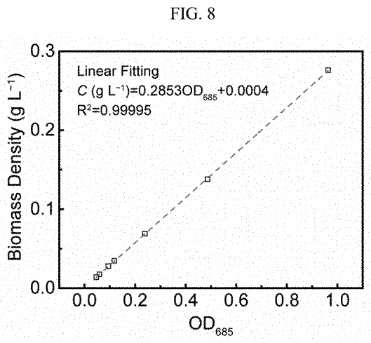

The sterilized flasks containing the microalgae were placed in a culture incubator (VWR International, Radnor, Pa.) with the temperature set at 22° C. and the light-to-dark time ratio of 1:1. The microalgal biomass density was determined by measuring the optical density at wavelength 685 nm (OD₆₈₅) using a UV-Vis spectrophotometer (Hach DR6000, Loveland, Colo.). All samples were properly diluted by BG-11 medium before the test to ensure the absorbance is within the range of 0.05-1. The obtained OD₆₈₅ values were then converted to biomass density (dry cell weight) based on the linear correlation: c (g L⁻¹)=0.2853OD₆₈₅+0.0004 (See FIG. 8). Microalgal suspension with an initial biomass density of ˜0.2 g L⁻¹ was used for all harvesting experiments unless otherwise specified.

### Preparation of PSAP Beads

To prepare PSAP beads, a reaction mixture containing ionic monomer (6 wt. % of sodium acrylate), non-ionic monomer (4 wt. % of acrylamide), and crosslinker (0.1% of N,N′-methylenebisacrylamide) was prepared. Poly(ethylene glycol) (PEG) with an average molecular weight of 6000 g mol⁻¹ was selected as the porogen and added to the reaction mixture (2.5-7.5 wt. %). Next, the initiator, 0.3 wt. % of ammonium persulfate, was added to the system and mixed well until fully dissolved. The 96-well plate with each well containing 15 μL of precursor solution was sealed and placed into a bath heater (Thermo Scientific, Waltham, Mass.) for 20 minutes at 70° C. The resultant polymer beads were washed with 95% ethanol to remove the porogen and then thoroughly dried in a 60° C. oven to obtain PSAP beads.

SAP beads, as the control group, were prepared using the same process but without the addition of PEG. The subsequent procedures including aliquoting and polymerization were similar. The reaction time for SAP fabrication was extended to 60 minutes for complete polymerization. The as-obtained SAP beads were then rinsed with DI water three times and dried at 60° C.

### Characterization of PSAP Beads

Imaging and analysis of the SAP and PSAP beads were performed by scanning electron microscopy (SEM, Hitachi SU8230, Tokyo, Japan). All specimens were coated with gold for 15 seconds at 20 mA by sputter coating (Quorum Q150T ES, Lewes, United Kingdom) prior to imaging. The swelling process of the SAP beads and PSAP beads prepared with different amounts of PEG (2.5-7.5 wt. %) in BG-11 medium was monitored for 1 hour using digital microscopy (Dino-Lite AM73915, Torrance, Calif.). During the swelling, the weight of the hydrated beads was measured and compared with the weight of the dried beads to calculate the swelling ratio (S=(m_{hydrated}−m_{dried})/m_{dried}). Three replicates were conducted for each kind of beads in the swelling kinetics test.

### Microalgae Harvesting Using the PSAP Beads

The performance of the PSAP beads for microalgae harvesting was tested. A certain amount of PSAP beads (m_{beads}) was added to a sterile tube with 5 mL of microalgal suspension (liquid volume: V₀, biomass density: c₀). The PSAP dosage was calculated based on the initial suspension volume and the dry bead weight, PSAP dosage (g L⁻¹)=m_{beads}/V₀. After 30 minutes absorption, the tube containing the microalgae and hydrated beads was gently shaken by hand to avoid aggregation and reduce biomass loss in subsequent separation. Next, the concentrated microalgae were separated from the hydrated beads using a pipet. The volume (V) and biomass density (c) of the concentrated liquid were measured to determine the harvesting efficiency, ρ(%)=cV/c₀V₀×100. Triplicate testing was performed for all harvesting conditions. The mean values and standard deviations of the obtained experimental results were calculated and reported. To investigate the microalgae adsorption on the PSAP beads, the surface and cross section of the beads after harvesting were visualized and analyzed by digital microscopy and fluorescence microscopy (Zeiss Axio Observer 7, Oberkochen, Germany). The viability of microalgal cells before and after PSAP treatment was determined by a flow cytometer (BD Accuri C6, San Jose, Calif.). The microalgae were stained by green fluorescent SYTO 9 nucleic acid dye (5 The excitation wavelength was 488 nm and fluorescence signals at 530 nm were measured to differentiate live and dead cells.

### Regeneration and Reusability of the PSAP Beads

The PSAP beads were applied to harvest 5 mL of microalgal suspension at an initial concentration of 0.2 g L⁻¹. After the treatment, the hydrated beads were separated and dewatered in a 60° C. oven for 2 hours. The regenerated PSAP beads were then used to treat another 5 mL of microalgal suspension. The absorption-desorption process of the PSAP beads was repeated five times (30 minutes absorption and 2 hours desorption). During the five cycles, the bead swelling ratio, microalgae harvesting efficiency, and concentration factor were monitored. After that, the PSAP beads were immersed in DI water for 1 hour to wash off the absorbed salt and reused for microalgae harvesting.

The description of the present embodiments of the invention has been presented for purposes of illustration but is not intended to be exhaustive or to limit the invention to the form disclosed. Many modifications and variations will be apparent to those of ordinary skill in the art. As such, while the present invention has been disclosed in connection with an embodiment thereof, it should be understood that other embodiments may fall within the spirit and scope of the invention.

All patents and publications cited herein are incorporated by reference in their entirety.

What is claimed is:

1. A method for harvesting microalgae from a medium, the method comprising
(a) adding a porous superabsorbent polymer bead to the medium;
(b) allowing water in the medium to absorb into pores in the porous superabsorbent polymer beads to form hydrated beads; and
(c) separating the microalgae from the hydrated beads, thereby harvesting the microalgae.

2. A method for increasing the concentration of microalgae in a medium, the method comprising
(a) adding a porous superabsorbent polymer bead to the medium;
(b) allowing water in the medium to absorb into pores in the porous superabsorbent polymer beads to form hydrated beads, thereby increasing the concentration of microalgae in the medium; and
(c) optionally, separating the microalgae from the hydrated beads.

3. The method according to claim 1, wherein the microalgae is Chlorella vulgaris, Isochrysis sp., Pseudioisochrysis sp., Dicrateria sp., Monochrysis sp., Tetraselmis sp., Pyramimonas sp., Micromonas sp., Chroomonas sp., Cryptomonas sp., Rhodomonas sp., Chlamydomonas sp., Chlorococcum sp., Olisthodiscus sp. Carteria sp., Dunaliella sp., Spirulina sp., Haematococcus sp., Rhodella sp., Arthrospira maxima, Nannochloropsis sp., or any combination thereof.

4. The method according to claim 1, where the microalgae are dried after separation from the hydrated beads.

5. The method according to claim 1, wherein in step (b) microalgae are excluded from the pores of the bead.

6. The method according to claim 1, wherein in step (b) microalgae are concentrated in the medium.

7. The method according to claim 1, wherein the medium is a microalgal suspension.

8. The method according to claim 1, wherein the porous superabsorbent polymer comprises a copolymer.

9. The method according to claim 8, wherein the copolymer comprises monomers selected from one or more ionic monomers, one or more non-ionic monomers, or any combination thereof.

10. The method according to claim 9, wherein the ionic monomers are selected from sodium acrylate, acrylic acid, potassium acrylate, itaconic acid, sodium itaconate, potassium itaconate, and any combination thereof.

11. The method according to claim 10, wherein the non-ionic monomers comprise acrylamide.

12. The method according to claim 1, wherein the pores in the superabsorbent polymer beads are between about 0.001 and about 10 micrometers.

13. The method according to claim 1, wherein the water absorbency of the superabsorbent polymer beads is between about 10 and about 150 g g⁻¹.

14. The method according to claim 1, wherein the superabsorbent polymer beads are made by a process comprising reacting one or more ionic monomers and/or one or more non-ionic monomers, or any combination thereof, with a porogen, optionally in the presence of a cross-linker.

15. The method according to claim 14, wherein the porogen is selected from poly(ethylene glycol), polyvinyl alcohol, or a combination thereof.

16. The method of claim 14, wherein the amount of porogen present in the monomer solution during preparation of the porous superabsorbent polymer beads is an amount between about 1 wt. % and about 10 wt. %.

17. The method according to claim 1, wherein the swelling ratio of the superabsorbent polymer beads is between about 1 g g⁻¹ and about 500 g g⁻¹.

18. The method according to claim 1, wherein the initial biomass density of the medium is between about 0.2 and about 70 g L⁻¹.

19. The method according to claim 1, wherein the final biomass density of the medium is between about 0.4 and about 150 g L⁻¹.

20. The method according to claim 1, wherein the efficiency of the process (harvesting efficiency) is greater than about 70%.

21. The method according to claim 1, wherein the method further comprises repeating steps (a) and (b) one or more times,

22. The method according to claim 21, wherein steps (a) and (b) are repeated, one, two, three, four or five times.
